(19) United States
(12) Patent Application Publication
(10) Pub. No.: US 2026/0345739 A1
(43) Pub. Date: Nov. 5, 2026

(54) STABLE INJECTABLE COMPOSITIONS OF GLP-2 PEPTIDE

(71) Applicant: ORBICULAR PHARMACEUTICAL TECHNOLOGIES PRIVATE LIMITED, Hyderabad (IN)

(72) Inventors: Patel Bhaveshkumar VALLABHBHAI, Hyderabad (IN); Raghu KANNEKANTI, Hyderabad (IN); Anjali Devi NIPPANI, Hyderabad (IN)

(21) Appl. No.: 19/739,190

(22) Filed: Jul. 13, 2026

Related U.S. Application Data
(63) Continuation of application No. 18/712,828, filed on Oct. 3, 2023.

(30) Foreign Application Priority Data
Oct. 3, 2022 (IN) 202241056675

(51) Int. Cl. A61K 38/26; A61K 9/00

(52) U.S. Cl. CPC A61K 38/26; A61K 9/0019

(57) ABSTRACT

The present invention relates to a stable GLP-2 peptide injectable composition, comprising GLP-2 peptide agent comprises of sodium; wherein the GLP-2 peptide agent is teduglutide or h[Gly2]GLP-2 and one or more pharmaceutically acceptable excipient(s).

### FIELD OF THE INVENTION

The present invention relates to a stable injectable composition, comprising active pharmaceutical ingredient is teduglutide comprises of sodium and one or more pharmaceutically acceptable excipient(s).

### BACKGROUND OF THE INVENTION

GLP-2 is a peptide secreted by L cells of the distal intestine. Like GLP-2, teduglutide is 33 amino acids in length. Teduglutide differs from native GLP-2 by a single amino acid substitution in position two, where alanine in GLP-2 is substituted by glycine in teduglutide. This substitution confers the peptide with resistance to in vivo degradation by the enzyme dipeptidyl protease-IV (DPP-IV).

The stability of the peptides is one of the critical aspect and various chemical and physical stability problems arises during storage. The peptides are more prone to degradation over time and undergo physical stability problems such as denaturation, adsorption, aggregation, precipitation, hence peptide molecules are difficult to formulate. Administration of peptides requires their formulation remain stable during the shelf life. The liquid formulation of peptide is more preferable but limited because of stability of peptides in liquid during shelf life. Therefore, to achieve the acceptable shelf life of peptide formulation there are various formulation approaches are adopted such as stabilization by structural modification, lyophilization, use of protein stabilizers, pH adjustment, storage under specific conditions, stabilization by variety of excipient(s), stabilizing buffers, salts, bases, counterions, metal ions, amino acids and the like.

Teduglutide is commercially available as lyophilized powder in a single-dose vial supplied with 0.5 mL sterile water for injection in a single-dose prefilled syringe for subcutaneous injection in a strength of 5 mg under the tradenames GATTEX and REVESTIVE. This is reconstituted with 0.5 mL of sterilised water for injection, i.e. after reconstitution a nominal 10 mg/mL solution is obtained and it is administered as subcutaneous injection via single-dose prefilled syringe. The teduglutide lyophilized powder is stored at 2 °C to 8 °C (36 °F to 46 °F) prior to dispensing and at room temperature up to 25 °C (77 °F) with a 90-day “use by” dating after dispensing by the pharmacist. Use GATTEX within 3 hours after mixing it. Each vial of Gattex contains 5 mg of teduglutide and additionally excipients 3.434 mg dibasic sodium phosphate heptahydrate, 3.88 mg L-histidine, 15 mg mannitol, and 0.644 mg monobasic sodium phosphate monohydrate. The L-histidine is used as stabilizer in the Gattex composition.

The Revestive (Shire/Takeda) product information in emc UK dated May 20, 2020; the composition comprises L-histidine as stabilizer and also includes the special warnings and precautions for use in label section 4.4 such as Revestive contains less than 1 mmol sodium (23 mg) per dose. This means that it is essentially ‘sodium-free’.

U.S. Pat. No. 7,056,886B2 is related to teduglutide lyophilised formulations for injection with improved stability and comprises phosphate buffer, L-histidine as stabilizer, bulking agents mannitol and sucrose.

U.S. Pat. No. 8,114,959 is related to a method for increasing the shelf-life of a pharmaceutical composition that comprises a glucagon-like peptide. The method comprises drying, preferably freeze drying, the peptide product at a pH above neutral pH.

U.S. Pat. No. 8,222,205 discloses a method by which an inactive GLP-1 compound can be converted into an active GLP-1 compound. The method comprises stirring the inactive compound in base (or acid) for a short period of time followed by neutralization and lyophilization. US′205 method specifically carried as Val-GLP-1 (7-37) OH was converted to soluble Val-GLP-1 (7-37) OH in high yield and without detectable racemization by dissolving in aqueous sodium hydroxide at pH 12.3, neutralizing to pH 7.0 and isolating the soluble product by filtration and lyophilization.

WO2022157747A1 is related to an aqueous stabilized peptide composition, said composition comprising a glucagon-like peptide such as teduglutide, liraglutide, a stabilizer L-histidine and a stabilizing peptide. It also discloses based stabilized teduglutide, prior to incorporation into formulation it is stabilized with 0.1 M ammonium hydroxide, lyophilize the mixture and then used in the formulation.

WO2019086559A1 is related to a stabilized composition of GLP-2, or an analogue thereof. The composition uses a co-polyamino acid having carboxylate charges and hydrophobic radicals as the stabilizing agent in order to prevent DPP-IV degradation of the polypeptide.

WO2022018748 is related to improved and effective purification processes for semaglutide. It also related to stable semaglutide comprising phosphate content in the range between 5% to 15% w/w and sodium content in the range between about 4% to about 10% w/w.

WO2021053578 is related to improved and effective purification processes and also relates to method of increasing the solubility of GLP-1 analogue and its derivatives particularly Liraglutide. It also related to liraglutide comprising 2.5-9.0% w/w of phosphate and 1.5-5.0% of sodium which is used for preparing pharmaceutical composition along with carriers.

The shelf life of injectable liquid formulation of peptide must be at least a year, preferably longer. The commercially available products having limited stability and requires special storage conditions. Thus, there in need in the art to develop injectable GLP-2 peptide composition with improved stability. The present invention provides stable injectable composition comprising GLP-2 peptide is teduglutide comprises of sodium. The sodium ions enhance the stability of GLP-2 peptide such as teduglutide which is used to formulate injectable solution composition along with pharmaceutically acceptable excipients with improved stability.

### SUMMARY OF THE INVENTION

The present invention relates to a stable injectable composition, comprising active pharmaceutical ingredient is GLP-2 peptide or analogue thereof comprises of sodium.

The present invention relates to a stable injectable composition, comprising active pharmaceutical ingredient is teduglutide comprises of sodium.

The present invention relates to a stable injectable composition, comprising active pharmaceutical ingredient is teduglutide comprises of sodium in an amount 2% w/w and above.

The present invention relates to a stable injectable composition comprising teduglutide comprises of sodium and one or more pharmaceutically acceptable excipient(s).

The present invention relates to a stable injectable composition comprising teduglutide comprises of sodium in an amount 2% w/w and above along with one or more pharmaceutically acceptable excipient(s); wherein the said composition is stable for longer duration of time.

The present invention relates to a process for preparing teduglutide API comprises sodium content and process for preparing stable injectable composition of teduglutide API comprises of sodium content along with one or more pharmaceutically acceptable excipient(s).

The present invention also relates to use of composition comprising teduglutide API comprises of sodium content along with one or more pharmaceutically acceptable excipient(s) for the treatment of gastrointestinal diseases or teduglutide sensitive diseases or disorders.

### DETAILED DESCRIPTION

The present invention relates to a stable injectable composition, comprising active pharmaceutical ingredient is GLP-2 peptide or analogue thereof comprises of sodium.

The present invention relates to a stable injectable composition, comprising active pharmaceutical ingredient is teduglutide comprises of sodium in an amount 2% w/w and above.

The present invention relates to a stable injectable composition, comprising active pharmaceutical ingredient is teduglutide comprises of sodium in an amount 2% w/w and above and one or more pharmaceutically acceptable excipient(s).

As used herein, the words or terms set forth below have the following definitions:

The terms “polypeptide”, “peptide”, and “protein” are used interchangeably herein to refer to polymers of amino acids of any length. The polymer may be linear or branched, it may comprise modified amino acids, and it may be interrupted by non-amino acids.

The term “active pharmaceutical ingredient” or “drug” or “API” refers to a substance that has a physiological effect when ingested or otherwise introduced into the body, in particular or a chemical substance used in the treatment, cure, prevention, or diagnosis of disease or used to otherwise enhance physical or mental well-being. The “active pharmaceutical ingredient” or “drug” or “API” is GLP-2 peptide or its analogue or its derivative or its metabolite or optical isomer, diastereomer, enantiomer, tautomer, or physiologically acceptable salt and solvates thereof.

The term “GLP-2 peptide” as used herein means GLP-2(1-33), a GLP-2 analogue, a GLP-2 derivative or a derivative of a GLP-2 analogue or any pharmaceutically acceptable salt thereof. Preferably GLP-2 peptide is teduglutide or h[Gly2]GLP-2.

The term “pharmaceutically acceptable” means approved by a regulatory agency or listed in the U.S. Pharmacopoeia or other generally recognized pharmacopoeia or formulary and the like for use in mammals, and more particularly in humans.

The term “pharmaceutically-acceptable salts” or “salts thereof” refer to derivatives of the disclosed compounds wherein the parent compound is modified by making acid or base salts, solvate, hydrate, esters and the like thereof. Pharmaceutically-acceptable salt forms of compounds provided herein are synthesized from the parent compound which contains a basic or acidic moiety by conventional chemical methods.

The term “amount” as used herein refers to quantity or to concentration as appropriate to the context.

The term “composition” or “formulation” or “preparation” is intended to encompass a combination including active ingredients and pharmaceutically acceptable excipients, as well as any product which results, directly or indirectly, from combination, complexation, or aggregation of any two or more of the ingredients, or from dissociation of one or more of the ingredients, or from other types of reactions or interactions involving one or more of the ingredients. The term “formulation” or “dosage form” or “composition” refers to finished pharmaceutical products that are suitable for administration, including, but not limited to, injections, etc.

The “injectable compositions” described herein include one or more active pharmaceutical ingredients. Any suitable active pharmaceutical ingredient can be employed, in any suitable amount, provided it effectively treats the disease or disorder in the subject. The amount of active pharmaceutical ingredient employed will typically depend on the active pharmaceutical ingredient as well as the disease or disorder to be treated.

The “stable injectable compositions” described herein include one or more active pharmaceutical ingredients with pharmaceutical acceptable excipient(s) having physical and chemical stability.

The “stable GLP-2 peptide injectable compositions” described herein include one or more GLP-2 peptide comprises of sodium with pharmaceutical acceptable excipient(s) having physical and chemical stability.

The term “parenteral” as used herein the compositions are administered by injection, infusion, or implantation into the human body. They may be directly administered or diluted before administration. The term “parenteral administration” as used herein is intramuscular, intraperitoneal, intra-abdominal, subcutaneous, intravenous, intraarterial, intradermal, intravitreal, intracerebral, intrathecal, epidural administration. In some embodiments, administration is subcutaneous.

The term “carrier” or “vehicle” or “solvent” or “diluent” or “excipient” or “excipient(s)” as used herein refers to pharmacologically inert materials that provide a more or less fluid matrix, suitable for parenteral drug administration. Carriers, vehicles or diluent useful herein include any such materials known in the art, which are nontoxic and do not interact with other components of a pharmaceutical formulation or drug delivery system in a deleterious manner.

The term “about” or “approximately” means within 10% of a given value or range or within an acceptable error range for the particular value as determined by one of ordinary skill in the art, which will depend in part on how the value is measured or determined, i.e., the limitations of the measurement system. Alternatively, the term “about” means within an acceptable standard error of the mean.

The term “comprising” or “comprises” or “including” or “having” or “containing” is inclusive or open-ended i.e. comprising what is specified in the present invention, but not excluding other or additional aspects, unrecited elements or method steps.

The term “further” or “additionally” as used herein, the unrecited elements, components may present along with the claimed cited element or components. It is to be construed as open-ended terms unless otherwise noted.

The term “optional” or “optionally” means that the subsequently described element, component or circumstance may or may not be present, so that the description includes instances where the element, component, or circumstance is included and instances where it is not.

The term “stability” or “stable” as used herein includes both physical and chemical stability. Stability parameters include but not limited to potency, stable pH value and other physico-chemical parameters. Peptide formulations are widely used as medicine. The most of the peptide drugs are formulated as liquid injectable compositions. The pharmaceutical compositions of such peptides are required to be stable or to have shelf life of several years in order to be suitable for use as medicine. However, peptide compositions are inherently unstable due to sensitivity towards physical and chemical degradation. Physical degradation involves transformations in the secondary, tertiary, or quaternary structure of the molecule or any conformational changes in the native structure of peptides, which may lead to aggregation, formation of fibrils, precipitation or surface adsorption. Chemical degradation change of covalent bonds, such as oxidation, hydrolysis, racemization or crosslinking. To ensure the products safety and efficacy, peptide formulation must meet defined quality and stability during and/or immediately after manufacture as well at the end of their designated shelf lives or during storage.

In one embodiment of present invention comprises GLP-2 peptide is teduglutide or (Gly2)GLP-2 or h[Gly2]GLP-2.

Teduglutide is a single-chain polypeptide with 33 amino acids having a mass of 3750.5 Da. It has no disulfide bonds, no glycosylation sites, and no post-translational modifications. Structural investigations of teduglutide show that the peptide contains varying amounts of α-helix and β-sheet secondary structures, depending on the peptide concentration and surrounding conditions. The sodium content in the teduglutide API enhances the stability of injectable solution.

In one embodiment of present invention provides a stable injectable composition, comprising:
a) active pharmaceutical ingredient comprises of sodium.

In one embodiment of present invention provides a stable injectable composition, comprising:
a) GLP-2 peptide as active pharmaceutical ingredient comprises of sodium.

In one embodiment of present invention provides a stable injectable composition, comprising:
a) GLP-2 peptide is teduglutide comprises of sodium.

In one embodiment of present invention provides, the GLP-2 peptide comprises of sodium.

In another embodiment of present invention provides, the GLP-2 peptide comprises of sodium in an amount 2% w/w and above.

In one embodiment of present invention provides, the Teduglutide comprises of sodium.

In another embodiment of present invention provides, the Teduglutide comprises of sodium in an amount 2% w/w and above.

In one embodiment of present invention provides, stable Teduglutide composition which comprises sodium in an amount 2% w/w and above.

Surprisingly, present inventors found that the Teduglutide comprises of sodium has improved physical stability specifically during storage. Importantly, improved physical stability of Teduglutide makes it more compatible for making injectable formulation and indirectly increasing the stability or shelf life.

In one embodiment of present invention provides a stable injectable composition, comprising:
a) active pharmaceutical ingredient comprises of sodium; and
b) one or more pharmaceutically acceptable excipient(s).

In one embodiment of present invention provides a stable injectable composition, comprising:
c) GLP-2 peptide is teduglutide comprises of sodium and
d) one or more pharmaceutically acceptable excipient(s).

In another embodiment of present invention provides teduglutide having sodium content in an amount 2% w/w and above, between the range of about 2% to about 20% w/w, about 2% to about 18% w/w, about 2% to about 16% w/w, about 2% to about 14% w/w, about 2% to about 12% w/w, about 2% to about 10% w/w, about 2% to about 8% w/w, about 2% to about 6% w/w, about 2% to about 4% w/w, about 2.2% to about 5.8% w/w, about 2.4% to about 5.6% w/w, about 2.6% to about 5.4% w/w, about 2.8% to about 5.2% w/w, about 3.0% to about 5.0% w/w, about 3.2% to about 4.8% w/w, about 3.4% to about 4.6% w/w, about 3.6% to about 4.4% w/w, about 3.8% to about 4.2% w/w, about 2% w/w, about 3% w/w, about 4% w/w, about 5% w/w, about 6% w/w, about 7% w/w, about 8% w/w, about 9% w/w, about 10% w/w, about 11% w/w, about 12% w/w, about 13% w/w, about 14% w/w, about 15% w/w, about 16% w/w, about 17% w/w, about 18% w/w, about 19% w/w, about 20% w/w or any value between the above ranges relative to the teduglutide.

In another embodiment of present invention provides a stable injectable composition comprising teduglutide comprises of sodium having pH is greater than 7.0. In another embodiments, the pH is in the range of 7 to 10, 7.5 to 9.5. In another embodiments, the pH is adjusted to about 7.0, 7.5, 8.0, 8.5, 9.0, 9.5, 10.0 or any pH value between the same.

In another embodiment of present invention, the teduglutide has a concentration of from 1 mg/mL to 25 mg/mL, from 2 mg/mL to 20 mg/mL, from 3 mg/mL to 15 mg/mL, from 4 mg/mL to 10 mg/mL, from 5 mg/mL to 10 mg/mL. In preferred embodiments, the concentration of teduglutide is about 1 mg/mL, about 2 mg/mL, 3 mg/mL, about 4 mg/mL, 5 mg/mL, about 6 mg/mL, 7 mg/mL, about 8 mg/mL, 9 mg/mL, about 10 mg/mL, about 15 mg/mL, about 20 mg/mL, or about 25 mg/mL.

In another embodiment, the pharmaceutical composition of present invention further comprises one or more pharmaceutically acceptable excipients such as tonicity adjusting agent, buffering agent, vehicle, carrier, pH modifiers, antioxidants, preservatives, chelating agents, bulking agents and lyoprotectants, diluents, surfactant, stabilizers, solvents and co-solvents, solubilizing, wetting, suspending, emulsifying or thickening agents, bulking agents, bases, counterions and the like or mixtures thereof. The choice of excipients depends on the desired characteristics of the compositions and on the nature of other pharmacologically active compounds in the formulation. Suitable excipients are known to those skilled in the art (see Handbook of Pharmaceutical Excipients, edited by Rowe et al, 6th edition, 2009).

Examples of tonicity adjusting agent or isotonic agents include but not limited to mannitol, sucrose, maltose, dextrose, trehalose, glycerin, sodium chloride, potassium chloride or mixtures thereof. Tonicity adjusting agents decrease the haemolysis of blood cells and reduce pain and irritation at the injection site.

Examples of buffering agent include but not limited to acetate, sodium acetate, acetic acid, glacial acetic acid, ammonium acetate, citrate, citric acid, sodium citrate, disodium citrate, trisodium citrate, tartrate, sodium titrate, tartaric acid, phosphate, phosphoric acid, monobasic potassium phosphate, dibasic potassium phosphate, monobasic sodium phosphate, sodium biphosphate, sodium dihydrogen phosphate, or sodium dihydrogen orthophosphate, dibasic sodium phosphate, sodium phosphate, disodium hydrogen phosphate, tribasic sodium phosphate, benzoate, benzene sulfonic acid, benzoate sodium/acid, lactate, gluconate, bicarbonate, organic amines, tromethamine/triethanolamine (tris), ammonium sulfate, ammonium hydroxide, arginine, aspartic acid, sodium bicarbonate, boric acid, sodium borate, sodium carbonate, diethanolamine, glucono delta lactone, glycine/glycine HCl, histidine/histidine HCl, hydrochloric acid, hydrobromic acid, lysine, maleic acid, meglumine, methanesulfonic acid, monoethanolamine, sodium hydroxide, succinate sodium/disodium, sulfuric acid and its salts, hydrates, solvates, or any mixtures thereof. In some embodiments, the pH of a pharmaceutical composition is controlled.

Examples of vehicle or carriers include but not limited to water for injection, sterile water for injection, hydroalcoholic solvents like, propylene glycol, polyethylene glycol, ethanol, glycerol, oils, triglycerides and mixtures thereof.

Examples of antioxidants include but not limited to monothioglycerol, thioglycerol, cystein/cysteinate hydrochloride, thioglycolic acid, thiourea, sodium metabisulfite, metabisulfite potassium, acetone sodium bisulfite, sulfite sodium, bisulfite sodium, ascorbic acid, sodium formaldehyde sulfoxylate, sodium bisulfate, butylated hydroxy toluene (BHT), butylated hydroxy anisole (BHA), argon, ascorbyl palmitate, phenylmercuric nitrate, thiomersal, benzalkonium chloride, benzethonium chloride, phenol, cresol or chlorobutanol, dithionite sodium (sodium hydrosulfite, sodium sulfoxylate), gentisic acid, gentisic acid ethanolamine, glutamate monosodium, glutathione, formaldehyde sulfoxylate sodium, methionine, nitrogen, propyl gallate, tocopherol alpha, alpha tocopherol hydrogen succinate, thioglycolate sodium, and the like or any mixtures thereof.

Examples of preservatives include but not limited to benzyl alcohol, m-cresol, phenol, methyl parabens, propylparaben, butylparaben, chlorobutanol, thiomersal, phenylmercuric salts, or mixtures thereof. The sterile solution of the present invention comprises preservative in amounts sufficient to maintain sterility of the solution in the injection device, throughout the shelf life of the product, which may be exposed to repeated multiple injections. This is because it is possible that the antimicrobial preservative concentration in a given preparation may decrease during the product's shelf life.

Examples of chelating agents include but not limited to ethylene diamine tetraacetic acid (EDTA) and acceptable salts thereof, citric acid, sodium citrate, ethylene glycol-bis(beta-aminoethyl ether)-N,N,N′,N′-tetraacetic acid (EGTA) and acceptable salts thereof, and 8-Amino-2-[(2-amino-5-methylphenoxy)methyl]-6-methoxyquinoline-N,N,N′,N′-tetraacetic acid, tetrapotassium salt (QUIN-2) and acceptable salts thereof and the like or mixtures thereof.

Examples of stabilizers include but not limited to polyols, amino acids, surfactants, poloxamer and its co-polymers; polyols include but not limited to propylene glycol, polyethylene glycol, polypropylene glycol and the like or mixtures thereof; amino acids include but not limited to aspartic acid, glutamic acid, glycine, arginine, lysine, L-histidine and the like or mixtures thereof; surfactants include but not limited to polysorbate-20, polysorbate-80, and the like or mixtures thereof.

Examples of surfactants include but not limited to polyoxyethylene sorbitan monooleate (tween 80), sorbitan monooleate polyoxyethylene sorbitan monolaurate (tween 20), polyoxyethylene polyoxypropylene copolymers (pluronics), Pluronic F-68, Lecithin, Polysorbate 20, Polysorbate 80, Sorbitan trioleate (span 85) and the like or mixtures thereof.

Examples of solvents and co-solvents include but not limited to propylene glycol, glycerin, ethanol, polyethylene glycol (PEG 300, PEG 400, PEG 600, PEG 3350, PEG 4000, PEG with any suitable molecular weight), sorbitol, dimethylacetamide and cremophor, benzyl benzoate, castor oil, cottonseed oil, N,N dimethylacetamide, ethanol/ethanol dehydrated, glycerin (glycerol), n-methyl-2-pyrrolidone, sulfoxides, peanut oil, poppyseed oil, safflower oil, sesame oil, soybean oil, vegetable oil, triglycerides, medium chain triglycerides and the like or mixtures thereof.

Examples of solubilizing agents include but not limited to Cyclodextrins, modified cyclodextrins, such as hydroxypropyl-β-cyclodextrin and sulfobutylether-β-cyclodextrin, phospholipids, phosphatidylcholine, soybean phosphatidylcholine, hydrogenated soybean phosphatidylcholine (HSPC), dimyristoyl phosphatidylcholine (DMPC), distearoyl phosphatidylcholine (DSPC), 1,2 dioleoyl-sn-glycero-3-phosphocholine (DOPC), distearoyl phosphoethanolamine (DSPE), L-alpha dimyristoyl phosphatidyl glycerol (DMPG), 1,2-dipalmitoyl-snglycero-3-phosho-rac-(1-glycerol), poloxamer or pluronic, polyethylene glycol 300, polyethylene glycol 400, ethanol, propylene glycol, glycerin, N-methyl-2-pyrrolidone, dimethylacetamide, dimethylsulfoxide, Cremophor EL, Cremophor RH 40, Cremophor RH 60, d-α-tocopherol polyethylene glycol 1000 succinate, polysorbate 20, polysorbate 80, Solutol HS 15, sorbitan monooleate, poloxamer 407, Labrafil M-1944CS, Labrafil M-2125CS, Labrasol, Gellucire 44/14, Softigen 767, mono- and di-fatty acid esters of PEG 300, 400, or 1750, castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil and palm seed oil, beeswax, d-α-tocopherol, oleic acid, medium-chain mono- and diglycerides, phospholipids (hydrogenated soy phosphatidylcholine, distearoyl phosphatidyl glycerol, I-α-dimyristoylphosphatidylcholine, I-α-dimyristoyl phosphatidyl glycerol) and the like or mixtures thereof.

Examples of bulking agents and lyoprotectants include but not limited to sucrose, lactose, trehalose, mannitol, sorbitol, glucose, raffinose, glycine, histidine, polyethylene glycol (PEG), PVP (K40), and the like or mixtures thereof.

Examples of base include but not limited to hydroxides such as ammonium hydroxide, sodium hydroxide, potassium hydroxide, calcium hydroxide, magnesium hydroxide; carbonates and/or bicarbonates such as sodium carbonate, potassium carbonate, ammonium carbonate, magnesium carbonate, phosphates such as sodium phosphate, potassium phosphate, ammonium phosphate, magnesium phosphate; amines and the like. In some embodiments the base may be organic and/or inorganic.

Examples of counterions include but not limited to ammonium, sodium, potassium, calcium, magnesium, and lithium and the like.

The present invention provides teduglutide composition having sodium content may optionally comprise other excipients known in the art to obtain a stable pharmaceutical composition.

In one embodiment provides methods or process for preparation of a stable injectable composition comprising, teduglutide comprises of sodium.

In one embodiment provides methods or process for preparation of teduglutide API comprises sodium.

In one embodiment provides methods or process for preparation of teduglutide API comprises sodium; wherein the sodium is added during the manufacturing process stage of teduglutide.

In one embodiment provides methods or process for preparation of teduglutide API comprises sodium; wherein sodium is added to the API prior to final composition.

In one embodiment provides methods or process for preparation of a stable injectable composition comprises the steps of addition of sodium in the form of base, buffer, counterion and/or salt before the final composition.

In one embodiment, the present invention provides method or process for preparation of a stable injectable composition comprising teduglutide having sodium content along with one or more pharmaceutically acceptable excipient(s).

In one embodiment, the present invention provides method or process for preparation of a stable injectable composition comprises the steps of
a) active ingredient comprises sodium content; and
b) admixing the said API comprises of sodium with a pharmaceutically acceptable excipient(s).

In one embodiment, the present invention provides method or process for preparation of a stable injectable composition comprises the steps of
c) the teduglutide as active ingredient comprises sodium content;

d) admixing the said teduglutide comprises of sodium with a pharmaceutically acceptable excipient(s).

In another embodiment of present invention teduglutide composition can be administered to patients in need of treatment therewith by any appropriate route. Preferably the composition can be administered by different parenteral routes such as but not limited to intradermal, subcutaneous, intramuscular, intravenous, intraperitoneal, intrathecal, epidural, intracardiac, intraarticular, intracavernous, intravitreal and the like.

In another embodiment of present invention provides stable injectable a composition can be administered by subcutaneous and/or intramuscular injection.

In one embodiment, the present invention compositions may be in the form of injectable dosage forms includes but not limited to suspension, solution, emulsion, nanosuspension, nanoemulsion, concentrated solutions for injections, ready to use or premix, ready to be dissolved in and/or diluted with a pharmaceutically acceptable vehicle, dry products ready to be dissolved in and/or diluted with a pharmaceutically acceptable vehicle, injectable microspheres, injectable microparticles, lyophilized powder for injection, spray dried powder for injection and the like.

In one embodiment, the present invention compositions may be in the form of aqueous and/or non aqueous injectable dosage forms.

In one embodiment, the present invention compositions comprise a solid (e.g., a powder) or a liquid solution that is diluted with a liquid carrier or diluent prior to administration to a subject.

In one embodiment, the present invention compositions may be in the form of lyophilized composition for reconstitution with one or more pharmaceutically acceptable diluents to provide a solution suitable for administration.

In another embodiment, the present invention provides lyophilized compositions comprising teduglutide sodium can be reconstituted by adding the pharmaceutically acceptable diluent(s) to provide the desired concentration for direct administration or further dilution for parenteral administration.

In one embodiment, the present invention injectable composition is prepared by the methods known in the art that maintains sterility, avoid the introduction of contaminants and microbial growth.

In one embodiment, the present invention compositions disclosed herein can be dispensed by suitable device such as autoinjector devices, prefilled syringes, injection pen, ampoules, vials, a glass vial, a plastic vial and the like.

In another embodiment of present invention compositions may be administered by self-administration injection device disclosed herein can be autoinjector, injection pen, prefilled syringe, pre-filled syringe fully assembled into an auto-injector device and the like.

The physical stability of Teduglutide for injection reconstituted solution were carried out against to the sodium content (%) present in Teduglutide API. The data is summarized in below table 1, 2 &3.

### Example: 1

TABLE 01
Reconstituted solution stability studies of teduglutide

| | | | Stability | |
|---|---|---|---|---|
| | | Sodium | of the | |
| | | (Na) | solution | |
| | | Content in | after | |
| Sr. | | Teduglutide | reconstitution | |
| No. | Batch No. | API | (hours) | Observation |
| 1. | Batch-1 | Nil | 1 hours | Hazy and |
| | | | | viscous |
| 2. | Batch-2 | 2.0 (%) | 5 hours | Hazy and |
| | | | | viscous |
| 3. | Batch-3 | 2.5 (%) | 8 hours | Hazy and |
| | | | | viscous |
| 4. | Batch-4 | 3.0% | 20 hours | Clear |
| 5. | Batch-5 | 4.0% | 20 hours | Hazy and |
| | | | | viscous |

The batches of teduglutide API comprises sodium were prepared according to the embodiment of the present invention. The reconstituted solution of teduglutide injection comprises sodium were monitored for physical and chemical stability. Teduglutide for injection after reconstitution shows hazy and viscous after certain time period as shown in table 01 (Batch 1 to 3 & 5). The teduglutide API comprises about 3% sodium content shows better stability than teduglutide API devoid or less sodium content.

Based on above experimental details, present inventors have considered batch 04 for further studies. Batch 4 of physical and chemical parameters are stable at 25 °C/60% RH for 6 months and 2 to 8 °C for 12 months after reconstitution.

### Example: 2

TABLE 02
Reconstituted solution stability studies of teduglutide

| | Batch no.: 4 | | | | | | | |
|---|---|---|---|---|---|---|---|---|
| Conditions | | 25 °C/60% RH | | | | 2-8 °C | | |
| Parameters | Initial | 1 M | 2 M | 3 M | 6 M | 3 M | 6 M | 12 M |
| Description | White Lyophilized cake | | | | | | | |
| pH | 7.44 | 7.51 | 7.48 | 7.52 | 7.49 | 7.5 | 7.52 | 7.5 |
| Osmolality | 272 | 281 | 282 | 275 | 278 | 288 | 270 | 272 |
| Teduglutide | 98.1 | 100.2 | 95.7 | 100.2 | 96.5 | 102 | 99.4 | 99 |
| assay (% w/v) | | | | | | | | |
| L-Histidine | 95 | — | 104 | 97.7 | 101.2 | 98.8 | 100.6 | 98.6 |
| content (%) | | | | | | | | |
| Total | 0.01 | 0.02 | 0.02 | 0.02 | 0.01 | 0.02 | 0.01 | 0.03 |
| aggregate | | | | | | | | |
| content (%) | | | | | | | | |

TABLE 03
Other related substance of batch 04

| | | Batch no.: 4 | | | | | | | |
|---|---|---|---|---|---|---|---|---|---|
| Conditions | | | 25 °C/60% RH | | | | 2-8 °C | | |
| Parameters | | Initial | 1 M | 2 M | 3 M | 6 M | 3 M | 6 M | 12 M |
| Description | | White Lyophilized cake | | | | | | | |
| Met (O)10 Impurity | | 0.13 | 0.17 | 0.13 | 0.13 | 0.1 | 0.11 | 0.11 | 0.15 |
| at RRT about 0.48 | | | | | | | | | |
| Iso-Asp3 Impurity at | | 0.3 | 0.29 | 30 | 0.28 | 0.28 | 0.28 | 0.28 | 0.29 |
| RRT about 0.91 | | | | | | | | | |
| Sum of Specified | | 0.2 | 0.29 | 0.25 | 0.37 | 0.35 | 0.2 | 0.1 | 0.21 |
| unidentified | | | | | | | | | |
| Impurity at RRT | | | | | | | | | |
| ~0.93 and Di-Gly4 | | | | | | | | | |
| impurity at RRT | | | | | | | | | |
| about 0.94 | | | | | | | | | |
| D-His1 Impurity at | | 0.12 | 0.08 | 0.13 | 0.12 | 0.14 | 0.13 | 0.14 | 0.18 |
| RRT about 0.96 | | | | | | | | | |
| Des-His1 Impurity at | | 0.03 | 0.04 | 0.08 | 0.1 | 0.05 | 0.1 | 0.05 | 0.06 |
| RRT about 1.03 | | | | | | | | | |
| Des-Gly2 Impurity at | | 0.06 | 0.03 | 0.03 | 0.03 | 0.05 | 0.03 | 0.05 | 0.06 |
| RRT about 1.06 | | | | | | | | | |
| Specified | about | 0.06 | 0.06 | 0.07 | 0.07 | 0.06 | 0.07 | 0.06 | 0.07 |
| unidentified | 0.62 | | | | | | | | |
| Impurity at | about | 0.06 | 0.09 | 0.03 | 0.04 | 0.06 | 0.03 | 0.05 | 0.1 |
| RRT | 0.98 | | | | | | | | |
| | about | 0.02 | 0.03 | 0.02 | 0.02 | 0 | 0.02 | ND | ND |
| | 1.02 | | | | | | | | |
| | about | 0.15 | 0.07 | 0.13 | 0.11 | 0.08 | 0.09 | 0.04 | 0.12 |
| | 1.05 | | | | | | | | |

| | | | | | | | | |
|---|---|---|---|---|---|---|---|---|
| Any unspecified | 0.07 | 0.09 | 0.07 | 0.07 | 0.06 | 0.07 | 0.05 | 0.06 |
| impurity | | | | | | | | |
| Total impurities | 1.25 | 1.21 | 1.27 | 1.34 | 1.29 | 1.13 | 0.93 | 1.3 |

Surprisingly, present inventors found that the Teduglutide comprises of sodium has improved physical stability specifically during storage. Importantly, improved physical stability of Teduglutide makes it more compatible for making injectable formulation and indirectly increasing the stability or shelf life.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A stable GLP-2 peptide injectable composition comprising:
GLP-2 peptide as active agent comprises of sodium in an amount 2% w/w and above;
wherein the GLP-2 peptide agent is teduglutide or h[Gly2]GLP-2; and
wherein the composition is physically stable during its shelf life.

2-8. (canceled)

9. The stable injectable composition of claim 1, wherein the reconstituted solution composition is physically stable at 25 °C/60% RH for 6 months.

10. The stable injectable composition of claim 1, wherein the composition is reconstituted solution composition is physically stable at 2 to 8 °C for 12 months.

11. The stable injectable composition of claim 9, wherein the reconstituted solution composition is physically stable at 25 °C/60% RH for 6 months and 2 to 8 °C for 12 months after reconstitution.

12. The stable injectable composition of claim 11, wherein the composition is stable upon storage at 25 °C/60% RH for 6 months and at 2 °C to 8 °C for 12 months, and wherein, reconstituted solution composition is physically and chemically stable, wherein the composition exhibits less than 2.0% total impurities.

13. The stable injectable composition of claim 1, wherein the GLP-2 peptide agent is teduglutide, and the composition comprises sodium and one or more pharmaceutically acceptable excipients.

14. The stable injectable composition of claim 13, wherein the composition further comprises one or more pharmaceutically acceptable excipient(s) selected from the group consisting of buffers, preservatives, antioxidants, surfactants, stabilizer, tonicity agents, bulking agents and combinations thereof.

15. The stable injectable composition of claim 1, wherein the composition is in the form of powder for reconstitution for injection.

16. The stable injectable composition of claim 1, wherein the composition is administered parenterally such as by subcutaneous injection.

17. The stable injectable composition of claim 1, wherein the composition is for use in the treatment of gastrointestinal diseases or teduglutide sensitive disease or disorders.
